(19) United States
(12) Patent Application Publication
(10) Pub. No.: US 2021/0361455 A1
(43) Pub. Date: Nov. 25, 2021

(54) ORTHOPAEDIC AID

(71) Applicant: Otto Bock Healthcare Products GmbH, Wien (AT)

(72) Inventors: Marcus EDER, Wien (AT); Wolfgang WAGNER, Wimpassing (AT); Josef INSCHLAG, Bruck an der Lafnitz (AT); Martin WASHÜTTL, Niederhollabrunn (AT); Martin WEBER, Groß-Enzerdorf (AT); Richard SEDLAK, Wien (AT); Roland AUBERGER, Wien (AT)

(73) Assignee: Otto Bock Healthcare Products GmbH, Wien (AT)

(21) Appl. No.: 17/053,738

(22) PCT Filed: May 6, 2019
(86) PCT No.: PCT/EP2019/061577

§ 371 Date: Nov. 6, 2020

(30) Foreign Application Priority Data
May 9, 2018 (DE) 10 2018 111 234.3

(51) Int. Cl. A61F 5/01; G01D 5/14; B25J 9/00

(52) U.S. Cl. CPC A61F 5/0123; G01D 5/147; B25J 9/0006; G01D 5/145

(57) ABSTRACT

An orthopaedic aid with a reference element, a movement element that is movably fixed to the reference element and a position sensor for determining a position of the movement element relative to the reference element, which comprises at least one permanent magnet and at least three Hall sensors, wherein the Hall sensors are arranged on the reference element and for moving along a trajectory upon a movement of the movement element relative to the reference element, wherein the at least one permanent magnet is fixed to the movement element and wherein the Hall sensors and the permanent magnet are arranged in such a way that a movement of the movement element relative to the reference element and a resulting movement of the Hall sensors along the trajectory causes a linear change in Hall voltage for at least one Hall sensor.

The invention relates to an orthopaedic aid. The invention also relates to an orthopaedic aid according to the general term in claim 2. According to a second aspect, the invention relates to a method for determining a position of such an orthopaedic aid.

Examples of orthopaedic aids include orthoses and prostheses, in particular exo-prostheses. Such orthopaedic aids often feature actuators, by means of which the properties of the orthopaedic aid are influenced depending on a position of the movement element relative to the reference element. It is therefore necessary to know the position of the orthopaedic aid, i.e. the position of the movement element relative to the reference element, to the highest possible degree of accuracy.

For weight reasons, orthopaedic aids generally do not have large energy stores, such that the determination of the position of the orthopaedic aid should also be conducted with as little energy as possible.

US 2014/0025182 A1 describes a prosthesis with a motor. The position of the motor is determined using a Hall sensor, which receives a signal when a magnetic element moves past it. The magnetic element is positioned on the rotor of the motor. The disadvantage of such orthopaedic aids is that a speed and acceleration of the movement element relative to the reference element can only be determined to a relatively imprecise degree.

US 2013/0245785 A1 describes a prosthesis with a vacuum pump. The vacuum pump can be operated with a switch that features a Hall sensor. If a magnetic element is guided past this Hall sensor, the switch switches. Such a system is only able to make a binary statement as to whether the switch has been switched or not.

US 2016/0302946 A1 describes a prosthesis with a load sensor. This load sensor comprises a Hall sensor and a magnet that moves relative to the Hall sensor when the prosthesis is subjected to a load. With such a system, it is indeed possible to determine the position of the two components of the prosthesis relative to one another relatively effectively; however, dynamic measurands, such as speed or acceleration, cannot be determined to a sufficient degree of accuracy.

US 2018/0116826 A1 describes a prosthesis which also features a Hall sensor, by means of which a position of a rotor of a motor relative to the stator can be determined. Such a system may also enable the determination of sufficiently accurate positional data, but not sufficiently accurate speed and/or acceleration data.

EP 2 696 814 B1 describes a prosthesis device which comprises a plurality of drives. It instructs that binary and non-binary sensors can be used, such as accelerometer or gyroscope. Such sensors are not well-suited to determining the relative position and relative speed between the movement element and reference element.

The invention aims to propose an orthopaedic aid whose position can be precisely determined.

The invention solves the problem by way of an orthopaedic aid with (a) a reference element, (b) a movement element that is movably fixed to the reference element and (c) a position sensor for determining a position of the movement element relative to the reference element, which comprises at least one permanent magnet and at least three Hall sensors, wherein (d) the Hall sensors are arranged on the reference element and for moving along a trajectory upon a movement of the movement element relative to the reference element, wherein (e) the at least one permanent magnet is fixed to the movement element and wherein (f) the Hall sensors are arranged in such a way that a movement of the movement element relative to the reference element and a resulting movement of the Hall sensors along the trajectory causes a linear change in Hall voltage for at least one Hall sensor.

The invention solves the problem by way of a method for determining the position of an orthopaedic aid with the properties stated above, with the steps (i) detecting a first Hall voltage of a first Hall sensor and a second Hall voltage of a second Hall sensor adjacent to the first Hall sensor, and (ii) determining the position of the movement element relative to the reference element from a Hall voltage difference between the first Hall voltage and the second Hall voltage and from the second Hall voltage. In particular, the position of the movement element is calculated from the Hall voltage difference and a distance between two adjacent Hall sensors and one of these Hall voltages. In particular, the determination is a calculation.

According to a second aspect, the invention also solves the problem by way of an orthopaedic aid with (a) a reference element, (b) a movement element that is movably fixed to the reference element, (c) a position sensor for determining a position of the movement element relative to the reference element, wherein the position sensor comprises at least one permanent magnet and at least three Hall sensors, and wherein (d) the Hall sensors are arranged on the reference element and the movement element is arranged to move along a trajectory relative to the reference element, wherein (e) the at least one permanent magnet features a first magnet arm, which extends in a magnet arm direction, and a second magnet arm, which is arranged at a distance from the first magnet arm and extends along the magnet arm direction, wherein (f) the first magnet arm has a first free end, which has a first magnetic polarity, wherein (g) the second magnet arm has a second free end, which has a second polarity that counters the first polarity, and wherein (h) the free ends are arranged along the trajectory and/or are movably arranged.

The advantage of such an orthopaedic aid is that its position, i.e. the position of the movement element relative to the reference element, can be determined to both a high degree of accuracy and with relatively little energy. Due to the high degree of measurement accuracy, it is also possible to determine by way of automatic derivation the speed at which the movement element moves relative to the reference element. Upon derivation, the measurement uncertainty increases considerably, which is why it is necessary to determine the position of the orthopaedic aid as precisely as possible. This is possible with the system according to the invention.

It is also advantageous that the determination of the position can be achieved in a robust manner. Hall sensors possess no movable parts, meaning there is little mechanical wear. Hall sensors are also standard components that are produced in large quantities to a high degree of accuracy. The orthopaedic aid is thus relatively cheap to produce.

Within the scope of the present description, an orthopaedic aid is understood particularly to mean a device that is designed to be connected to a human or animal body in order to replace or support the function of a diseased or non-existent joint or the muscles surrounding the joint.

The orthopaedic aid is understood in particular to refer to an orthosis or prosthesis, especially an exo-prosthesis. For example, the orthopaedic aid is a knee exo-prosthesis.

The reference element is understood in particular to mean a component of the aid in relation to which the movement element performs a relative movement. It should be noted that this refers to a relative movement of the movement element to the reference element. As such, the movement element could also be understood as a reference element and the reference element as a movement element. The terms are merely intended to facilitate the explanation of the invention. Given that only the relative movements between the reference element and the movement element are of significance, the element to which the Hall sensors are fixed is considered the reference element. The term first element could also be used instead of reference element and the term second element instead of the term movement element.

The feature that the movement element is movably fixed to the reference element is understood particularly to mean that they are attached to one another in a defined guided manner.

The position sensor is understood particularly to mean any device by means of which the position of the movement element relative to the reference element can be automatically determined. Preferably, the position sensor emits an electrical signal that encodes the position of the movement element relative to the reference element. Here, it is possible, but not essential, for this electrical signal to specify the position in absolute units, especially SI units. In particular, it is also possible that the position is given in a coordinate and/or unit system that is specific to the orthopaedic aid.

The feature that the Hall sensors are arranged on the movement element to move along a trajectory to move the movement element relative to the reference element is understood particularly to mean that a movement of the movement element towards a reference element that is considered stationary causes the Hall sensors to move along a curve, namely the trajectory. In particular, all Hall sensors move along the same trajectory. The trajectory may refer, for instance, to a circle or a straight line; however, this is not necessary. In particular, the trajectory may also be an ellipse, for instance, or another form.

The feature that the Hall sensors are arranged in such a way that a movement of the Hall sensors along the trajectory effects a linear change in Hall voltage for at least one Hall sensor is understood particularly to mean that there is always one Hall sensor to which this claim applies. In particular, this claim does not generally apply for all Hall sensors at once; rather, it only applies for two Hall sensors, regardless of the position of the movement element relative to the reference element. A linear change in Hall voltage is understood to mean a linear change in the technical sense. In other words, it is possible that the Hall voltage is not mathematically linearly dependent on the change in position of the Hall sensor as long as this deviation is sufficiently small.

Of course, any curve can generally be regarded as linear at first approximation, but this is not what is meant by the present feature. Rather, the Hall sensors are arranged such that a measurement error, which is caused by assuming the linearity of the change in Hall voltage as a function of a change in position, is smaller than a predetermined value, which is preferably less than 2%.

It is especially beneficial if the Hall sensors are temperature-compensated.

The feature that the first magnet arm extends in the magnet arm direction is understood particularly to mean that the first magnet arm extends in this direction adjacent to its free end. If the magnet arm is prismatic, in particular cuboid in shape, the magnet arm direction corresponds to the translation direction of the prism.

The feature that the free ends, mounted on the movement element, are arranged or move along the trajectory is understood particularly to mean that both ends are the same distance from the trajectory. As is standard practice, the distance is understood to mean the length of the shortest distance that connects two objects to each other. The same distance is understood to mean the same distance in the technical sense. It is therefore possible, but not essential, for the distance to be the same in the mathematical sense; however, relative deviations of, for instance, a maximum of 10% are also possible.

According to a preferred embodiment, the permanent magnet has (a) a magnetic flux forming part comprising a soft magnetic element made of a soft magnetic material, (b) a first partial permanent magnet, which forms the first magnet arm and rests on the soft magnetic element with its first contact end opposite the first free end, (c) a second partial permanent magnet, which forms the second magnet and rests on the soft magnetic element with its second contact end opposite the second free end and (d) a third partial permanent magnet, which is arranged between the first partial permanent magnet and the second partial permanent magnet, extends transversely to the first magnet arm and the second magnet arm and has a magnetic third permanent magnet orientation which extends transversely to a first permanent magnet orientation of the first partial permanent magnet and extends transversely to a second permanent magnet orientation of the second partial permanent magnet.

A permanent magnet constructed in this way has been proven to generate an especially homogeneous field at the point of the Hall sensors.

Preferably, the magnetic flux forming part comprises a non-ferromagnetic part which is arranged in the magnetic flux line curve between the first partial permanent magnet and the third partial permanent magnet and/or which is arranged in the magnetic flux line curve between the second partial permanent magnet and the third partial permanent magnet. The feature that the non-ferromagnetic part is arranged in the magnetic flux line curve between the first and the third partial permanent magnet is understood particularly to mean that the magnet flux lines extend from the first partial permanent magnet through the non-ferromagnetic part to the third partial permanent magnet. The non-ferromagnetic part is composed of material, especially diamagnetic or paramagnetic material, that is not ferromagnetic, such as a metal, in particular copper, or plastic. The thickness of the non-ferromagnetic part is selected in such a way that the Hall sensors exhibit an approximately linear change in Hall voltage across the widest possible range of movement along the trajectory for at least one Hall sensor. The ideal thickness is determined during pre-trials.

Preferably, the permanent magnet has a permanent magnet length along the trajectory that is at least twice as great, especially three times as great, as a Hall sensor distance between two adjacent Hall sensors. This results in a sufficiently homogeneous magnetic field at the point of the Hall sensor, so that a high degree of measurement accuracy can be achieved.

The distance between two Hall sensors is understood particularly to mean the distance by which the Hall sensors must be moved until the adjacent Hall sensor is arranged at the same point as the previous Hall sensor.

Preferably, a distance of the trajectory from the permanent magnet is at most half of the permanent magnet length. This causes a sufficiently homogeneous magnetic field in the Hall sensors. It is also practical if the distance is at least one tenth of the permanent magnet length.

The orthopaedic aid preferably features an electric evaluation unit that is designed to automatically carry out a method comprising the steps (i) detecting a first Hall voltage of a first Hall sensor and a second Hall voltage of a second Hall sensor adjacent to the first Hall sensor, and (ii) determining a position of the movement element relative to the reference element from a Hall voltage difference between the first Hall voltage and the second Hall voltage.

The Hall voltages applied to the Hall sensors could already be used to determine the position of the Hall sensors relative to the permanent magnet and thus the position of the movement element relative to the reference element. However, it has been found that the additional consideration of the Hall voltage difference allows the position of the movement element relative to the reference element to be determined with greater accuracy.

Preferably, the electric evaluation unit is designed to automatically carry out a method featuring the steps (i) detecting the Hall voltage of at least three Hall sensors, (ii) detecting those Hall sensors for which the Hall voltages assume the smallest values in terms of magnitude and (iii) determining the position of the movement element relative to the reference element from the positions of these Hall sensors and a Hall voltage difference of these Hall voltages. With correctly positioned Hall sensors, the Hall voltage disappears if the applied magnetic field does not have any normal components on the sensor plane. This is preferably the case if the Hall sensor is situated exactly between the two magnet arms. A deviation from this position causes a linear change of Hall voltage to a good approximation. A linear change to a good approximation is understood to mean that the deviation from linear behaviour is at most 2%.

To calculate the Hall voltage difference to the adjacent Hall sensor, the Hall voltage that is the smallest in terms of magnitude is preferably used. This voltage belongs to the Hall sensor whose distance from the position specified above between the two magnet arms is smaller than that of the other adjacent Hall sensor. This ensures that, for determining the position of the orthopaedic aid, the two Hall sensors which are situated the shortest distance away from the position between the two magnet arms are used. This enables an especially high degree of accuracy when measuring the position.

It is beneficial if the reference element is a cylinder and the movement element is a piston that is inside the cylinder, wherein the position sensor is a piston position sensor for measuring a position of the piston in the cylinder, and wherein the permanent magnet is arranged on the piston and the piston position sensor is arranged on the cylinder. This allows the position of the piston in the cylinder to be measured with greater accuracy.

Alternatively or additionally, the reference element is a first limb, the movement element a second limb, wherein the first limb and the second limb are connected by means of a joint, in particular a swivel joint, and the position sensor is a limb angle sensor for measuring the angular position of the first limb relative to the second limb.

According to a preferred embodiment, the evaluation unit is designed to switch off Hall sensors whose measurement results are not taken into account when calculating the position of the movement element. This keeps energy consumption at a low level.

The invention also includes a method for determining a position of an orthopaedic aid with a reference element, (b) a movement element, (c) a position sensor for determining a position of the movement element relative to the reference element, which comprises at least one permanent magnet and at least three Hall sensors, (d) wherein the Hall sensors are arranged on the reference element and the movement element is arranged to move along a trajectory relative to the reference element, featuring the steps: (i) detecting a first Hall voltage of a first Hall sensor and a second Hall voltage of a second Hall sensor adjacent to the first Hall sensor, and (ii) determining the position of the movement element relative to the reference element from a Hall voltage difference between the first Hall voltage and the second Hall voltage and the second Hall voltage.

The method preferably comprises the steps: (i) detecting the Hall voltage of at least three Hall sensors, (ii) detecting those Hall sensors, especially two Hall sensors, for which the Hall voltages assume the smallest values in terms of magnitude and (iii) determining the position of the permanent magnet from the positions of these Hall sensors and a Hall voltage difference of these Hall voltages. If two Hall voltages are the same, one of the two Hall sensors is selected, for instance the sensor with a lower index number, wherein in this case, all Hall sensors have an index number and are arranged according to the size of the index number.

The method preferably comprises the steps: (i) for each Hall sensor, detecting an offset voltage of the Hall voltage, which is caused by the existence of an angle between an actual position of the Hall sensor and a nominal position, and (ii) correcting the Hall voltage by the offset voltage. In particular, the nominal position is the one in which no Hall voltage is applied to the Hall sensor when the Hall sensor is situated exactly between the two magnet arms. If the Hall sensor is mounted at a tilt in relation to this nominal position, a normal component of the magnetic field also occurs in this position. This normal component is the same one which would result from a movement along the trajectory. It is therefore advantageous to subtract this offset voltage from the measured Hall voltage. To carry out this correction, the electric evaluation unit preferably has a digital memory in which the offset for each Hall sensor is stored and the evaluation unit is designed to automatically subtract the offset voltage from the measured Hall voltage.

This offset voltage is measured, for instance, by measuring the voltage when no magnetic field is applied to the Hall sensor. The Hall voltages U_{Hall,N} measured during subsequent use are corrected by the value of the offset voltage during evaluation. U′_{Hall,N} corresponds to the thus corrected value of the Hall voltage.

According to a preferred embodiment, the method comprises the steps (i) for each Hall sensor, detecting a sensitivity that describes the dependency of the Hall voltage on the magnetic field, and (ii) correcting the position by the influence of the sensitivity. The sensitivity is measured by applying a known magnetic field to the Hall sensor and measuring the resulting Hall voltage. The sensitivities measured in this way are stored digitally in the evaluation unit for all Hall sensors.

For instance, the Hall sensors are calibrated in a testing machine. In this case, a digital incremental encoder is flange-mounted to a motor. The motor moves a reference magnet in a circle across the Hall sensors. The incremental encoder provides the exact topical angle value, the sensors provide the Hall voltages.

In this case, the curves for all Hall sensors 42.i are recorded. The sensitivity is the slope of the curve that applies the Hall voltage against the magnetic field.

In the following, the invention will be explained in more detail by way of the attached figures. They show

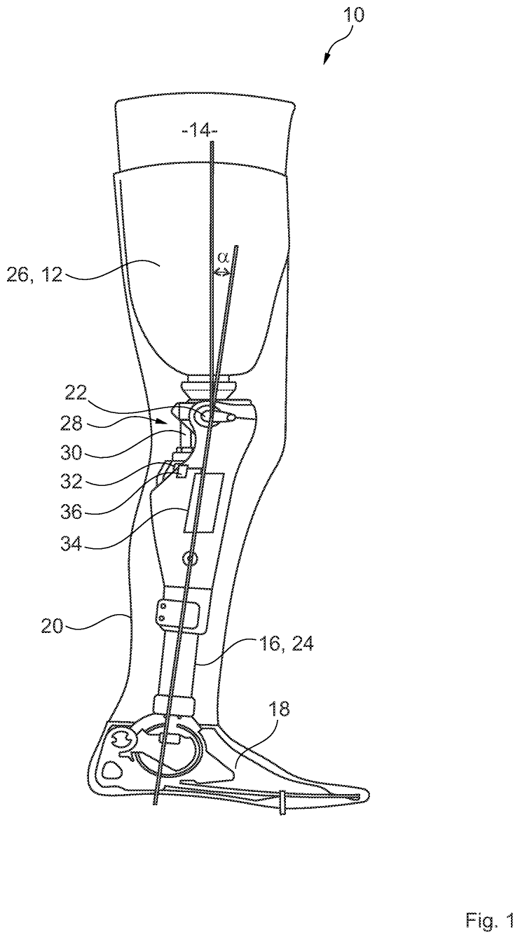

FIG. 1 a side view of an orthopaedic aid according to the invention in the form of a knee exo-prosthesis,

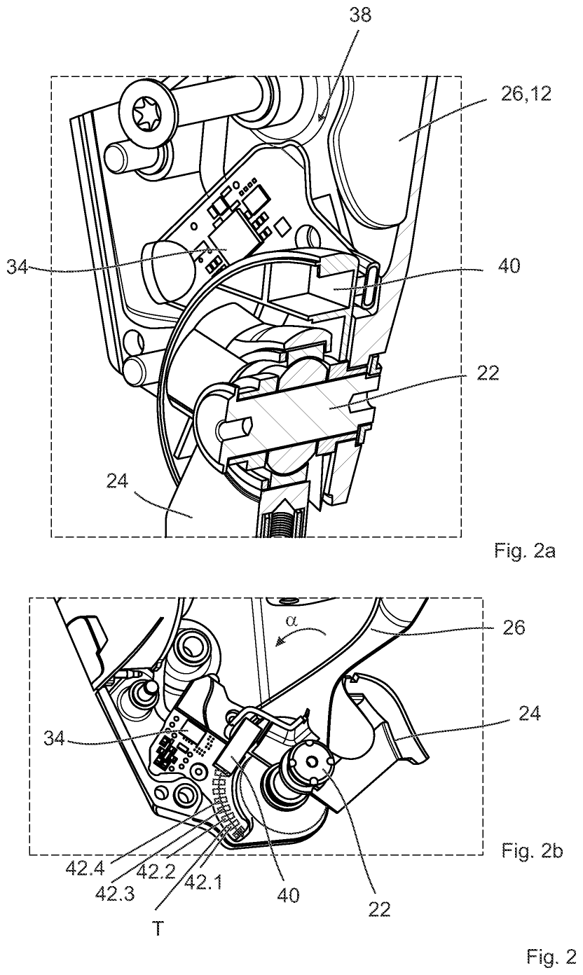

FIG. 2a a section of the orthopaedic aid according to FIG. 1 which contains the reference element, the movement element and the position sensor,

FIG. 2b the orthopaedic aid according to FIG. 2a where the movement element has been partially removed,

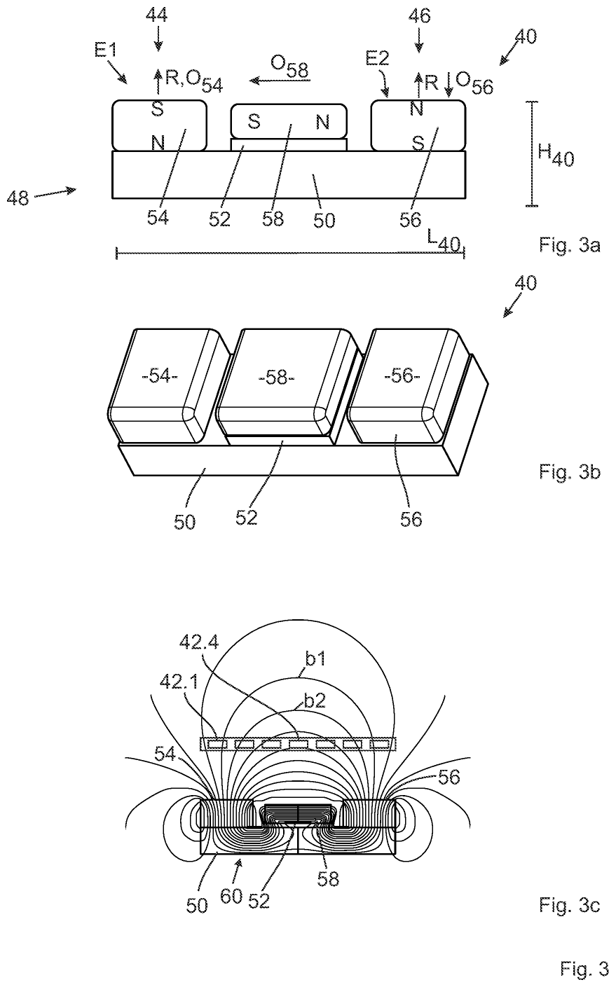

FIG. 3a a schematic side view of the permanent magnet of the orthopaedic aid according to FIGS. 1 and 2,

FIG. 3b a perspective view of the permanent magnet according to FIG. 3a,

FIG. 3c the magnetic line of the permanent magnet according to FIGS. 3a and 3b,

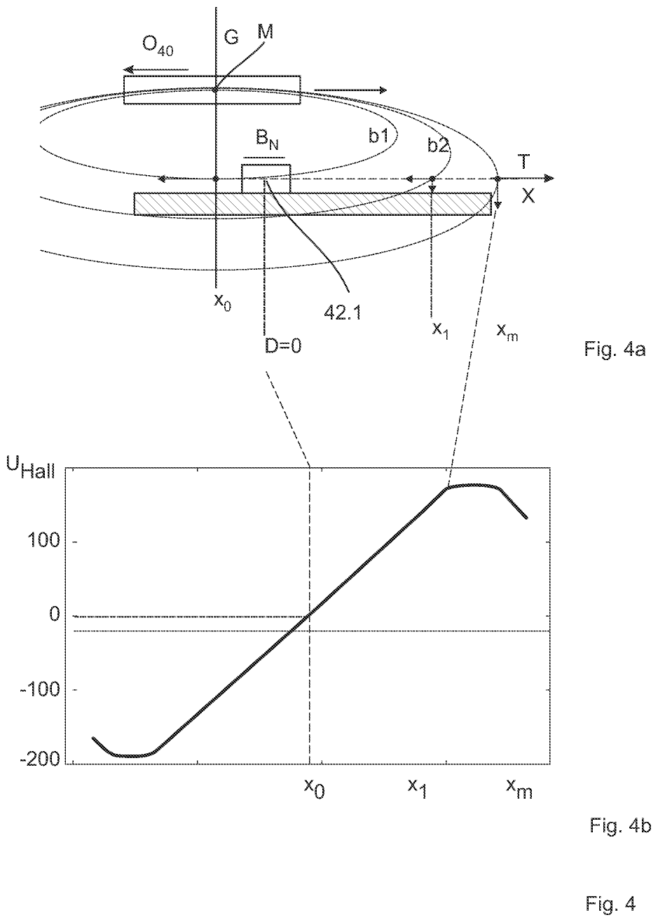

FIG. 4a a view of the magnetic line curve of the permanent magnet in relation to a Hall sensor,

FIG. 4b the dependence of the Hall voltage on a position of a Hall sensor relative to the permanent magnet,

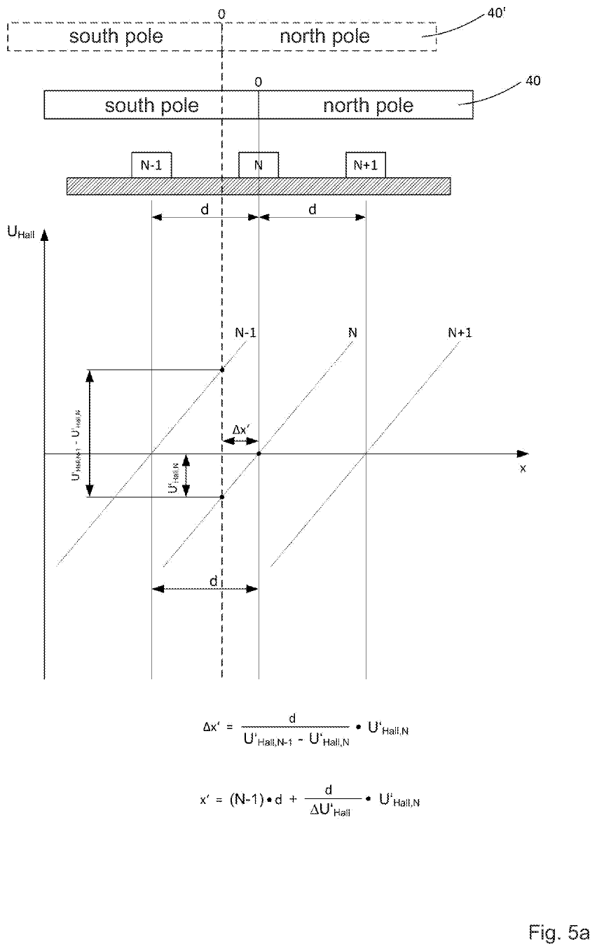

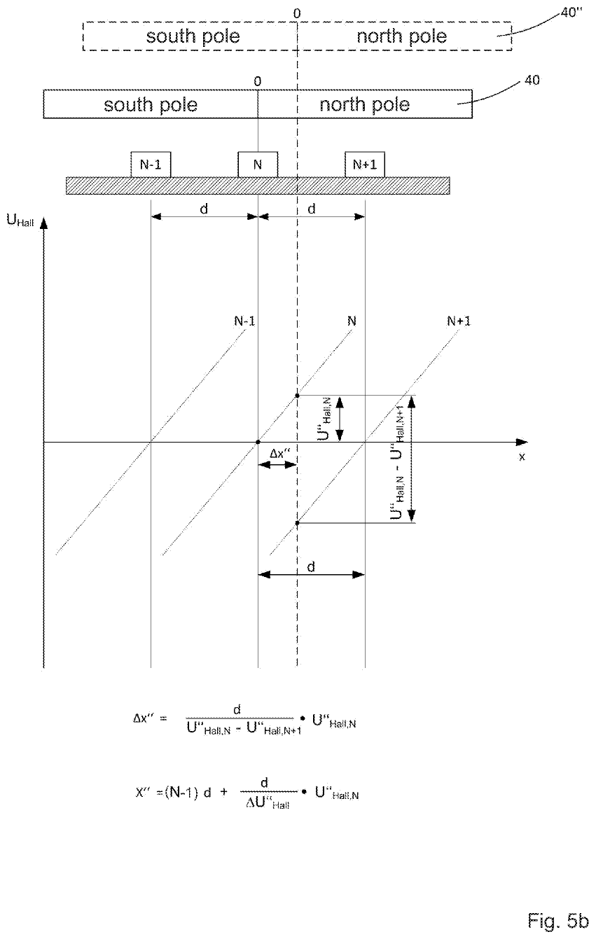

FIG. 5a the Hall voltage curves of three Hall sensors to explain the determination of the position of the Hall sensors relative to the permanent magnets,

FIG. 5b the curve of the Hall voltages for several Hall sensors as a function of the position of the movement element relative to the reference element to explain the calculation of the position, and

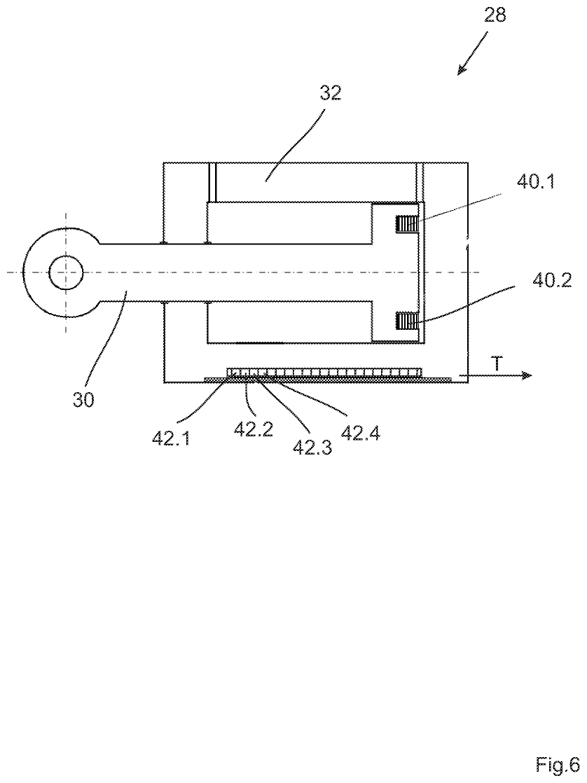

FIG. 6 a cylinder of an orthopaedic aid according to the invention, the position sensor of which is designed to determine the position of the cylinder in the piston.

FIG. 1 shows an orthopaedic aid 10 according to the invention in the form of a knee exo-prosthesis that comprises a shaft 12 for accommodating a human upper leg stump 14 and an artificial lower leg 16. The lower leg 16 is connected to an artificial foot 18. It is possible and preferable for the orthopaedic aid to have a cosmetic cover 20, which lends the knee exo-prosthesis a natural appearance.

The aid 10 features a swivel joint 22, about which the lower leg 16 can swivel relative to the shaft 12 at a swivel angle α. In the present case, the shaft 12 represents a reference element 26, relative to which a movement element 24 in the form of the lower leg can move.

In the present case, the aid 10 features a damper 28 which has a piston 30 that is inside a cylinder 32. Depending on the position of the movement element 24 relative to the reference element 26, the position of the piston 30 in the cylinder 32 changes.

The orthopaedic aid 10 according to FIG. 1 makes it clear that only a relative movement between the movement element 24 and the reference element 26 is relevant. In addition, the reference element 26 also moves during use of the aid 10.

The aid 10 comprises a schematically depicted evaluation unit 34 that is possibly, but not necessarily, connected to a schematically depicted actuator 36. The actuator 36 can be used to change the damping properties of the damper 28. In particular, the damper can preferably be locked, so that the piston 30 can no longer move in the cylinder 32. Alternatively or additionally, the actuator can be used to change the force that must be applied to the piston 30 in order to move it relative to the cylinder 32 at a predetermined speed.

FIG. 2a shows a cut partial view of the aid 10. It should be noted that the aid 10 features a position sensor 38. The position sensor 38 comprises the Hall sensors 42.i (i=1, 2, . . . , 18) and the evaluation device 34, which is fixed to the reference element 26 (not visible in FIG. 2a). A permanent magnet 40 of the position sensor 38 is fixed to the reference element 24.

FIG. 2b depicts the position sensor in detail. The position sensor 38 comprises the permanent magnet 40 and the Hall sensors 42.i (i=1, 2, . . . , 18) as well as the evaluation device 34. The Hall sensors 42.i are fixed to the reference element 26, in this case rigidly relative to shaft 12. Conversely, the permanent magnet 40 is fixed to the movement element 24, in this case rigidly relative to lower leg 16.

If the movement element 24 moves relative to the reference element 26, the Hall sensors 42.i move relative to the movement element 24 on a trajectory T. In the present case, the trajectory T is an arc. In the present case, the trajectory T depends on the swivel angle α (cf. FIG. 1) between the movement element 24 and the reference element 26.

FIG. 3a shows a side view of the permanent magnet 40. The permanent magnet 40 has a first magnet arm 44, which extends in a magnet arm direction R. The permanent magnet 40 also has a second magnet arm 46, which also extends along the magnet arm direction R. The first magnet arm 44 has a first free end E1 with a first polarity P1, this being the south pole in the present case. The second magnet arm 46 has a second free end E2 with a second polarity P2 that is opposite the first polarity P1; in the present case, therefore, a north pole.

The permanent magnet 40 also has a magnetic flux forming part 48 that comprises a soft magnet element 50 and a non-ferromagnetic part 52. In the present embodiment, the soft magnet element 50 is composed of soft iron; in the present case, the non-ferromagnetic part 52 is made of copper. For instance, the non-ferromagnetic part 52 could also be made of plastic and acts, in particular, as a spacer.

FIG. 3a shows that the permanent magnet 40 features a first partial permanent magnet 54, a second partial permanent magnet 56 and a third partial permanent magnet 58. The first partial permanent magnet 54 forms the first magnet arm 44 and the second partial permanent magnet 56 the second magnet arm 46.

The third partial permanent magnet 58 is arranged between the first partial permanent magnet 54 and the second partial permanent magnet 56 and extends transversely to them. In other words, a third permanent magnet orientation O₅₈, which extends from the north pole to the south pole, extends transversely to a first permanent magnet orientation O₅₄, which corresponds in the present case to the magnet arm direction R. The third permanent magnet orientation O₅₈ also extends transversely to a second permanent magnet orientation O₅₆, which in the present case extends in the opposite direction to the magnet arm direction R. The feature that the third permanent magnet orientation O₅₈ extends transversely to the first permanent magnet orientation is understood particularly to mean that an angle between the two is at least predominantly 90°. That is to say that the angle lies between 85 and 95°.

FIG. 3a shows that a height H₄₀ of the permanent magnet 40 is smaller than its length L₄₀. The height H₄₀ is measured in the direction of the magnet arm direction R. The length L₄₀ is preferably twice as great as the height H₄₀, preferably at least 2.5 times as great.

FIG. 3a shows a perspective view of the permanent magnet 40. The partial permanent magnets 54, 56, 58 and the moulded magnet flux part 48 are connected to one another, for example they are stuck together.

FIG. 3c depicts a magnetic flux line curve 60 of the magnetic flux lines b1, b2, . . . . It should be noted that a normal component B_{N} of the magnetic field at the point of the Hall sensor 42.4, which is situated exactly between the two ends E1 and E2, disappears.

FIG. 4a shows the curve of the magnetic flux lines b1, b2, . . . , if a rod magnet is used as a permanent magnet rather than the permanent magnet as it is depicted in FIGS. 3a, 3b and 3c. Each Hall sensor 42, such as the Hall sensor 42.1, produces a Hall voltage U_{Hall} according to FIG. 4b, which depends on the normal component B_{N} of the magnetic field B in relation to a sensor plane E of the Hall sensor 42.1. A normal component B_{N} does not exist on a straight line G, which extends perpendicular to the magnet orientation O₄₀ through a centre point M of the permanent magnet 40. Correspondingly, the Hall voltage Ulla at this point, which is described as xₒ, is equal to zero.

If the permanent magnet 40, which is fixed to the movement element, moves, the Hall sensor 42.1 moves along the trajectory T, in the present case a straight line, relative to the movement element. The permanent magnet 40 thus also moves relative to the reference element along the trajectory T. As a result, the Hall voltage U_{Hall} initially changes linearly and passes through a maximum at a point x_{M}. The reason for this is that, although the angle between the magnetic field line and the sensor plane is constantly increasing, the magnetic field becomes smaller with distance in the third power.

FIG. 4b depicts the dependency of the Hall voltage U_{Hall} on the position of the Hall sensor.

FIG. 5a depicts the dependency of the Hall voltage Ulla on an x-coordinate along the trajectory T. The upper part of the image shows the positions of the generally defined Hall sensors 42.N, 42.N+1 and 42.N−1. The lower part of the image shows that the slope k=ΔU_{Hall}/d can be determined as

$$k := \frac{{U}_{Hall , N - 1} - {U}_{Hall , N}}{d} = \frac{{Δ U}_{Hall}}{d}$$

d is the distance between two Hall sensors, for example the Hall sensors 42.N and 42.N+1. ΔU_{Hall} is the Hall voltage difference. The Hall sensors 42.i are arranged to be equidistant, meaning that the distance between to adjacent Hall sensors is always the same d.

If the permanent magnet is displaced, for instance to the position shown by the dashed line, the voltage curve is also displaced. The Hall voltage U′_{Hall,N} is the result of the displacement Δx′ along the trajectory T, wherein the Hall sensor 42.N measures said Hall voltage following the displacement by Δx′ to

$${{U}^{′}}_{Hall , N} = {k Δ x}^{′} = \begin{matrix}{{Δ U}^{′}}_{Hall} \\ d\end{matrix} {Δ x}^{′}$$

The measured Hall voltage U′_{Hall,N} can thus be used to determine the position of the Hall sensor 42.N and therefore the position of the reference element 26 relative to the movement element 24 (cf. FIG. 2). There are generally more than three Hall sensors 42.i.

FIG. 5b features the dependencies of the Hall voltage U_{Hall,i} for several Hall sensors. If the permanent magnet 40 is in the position Δx″ relative to the Hall sensors, as shown by the dashed line in FIG. 5b (and indicated with permanent magnet 40″), the specified Hall voltages U″_{Hall,N-1}, . . . , U″_{Hall,N+2} are measured.

A first step comprises the determination of the Hall voltages that lie closest to the value that is measured at the position shown in FIG. 5b for the Hall sensor 42.n, namely at which the Hall sensor is arranged exactly between two two magnetic poles of the permanent magnet 40. This value is generally U″_{Hall}=0 V, given that no normal magnetic field component exists for the corresponding Hall sensor.

Therefore, in the present case, the three smallest Hall voltages U″_{Hall} in terms of magnitude are determined. This refers to the Hall voltages U″_{Hall,N-1}, . . . , U″_{Hall,N+1}. The smallest value in terms of magnitude is U″_{Hall,N}. The next-smallest Hall voltage in terms of magnitude is U_{Hall,N+1.}

Therefore, the following applies:

U″_{Hall,N}=+kΔx″.

The position is therefore

$${x}^{″} = ( N - 1 ) d + \frac{{{Δ U}^{″}}_{Hall}}{k} .$$

In this formula, x″ is the path along the trajectory T, wherein x=0 at the point of the first Hall sensor 42.1.

If the permanent magnet 40 continues to move, the voltage U″_{Hall,N}, for instance, continues to increase until it is greater in terms of magnitude than the voltage U″_{Hall,N+1}. At this point, the calculation with the small Hall voltage in terms of magnitude is conducted. It should be noted that the smallest voltages in terms of magnitude always refer to the voltage of the Hall sensor that is arranged like the Hall sensor 42.N in FIG. 5a, i.e. in the area exactly between the two free ends of the permanent magnet 40. This voltage is generally zero.

It is possible that this voltage is not zero but rather an offset voltage U_{Offset}, for example as a result of a tilted assembly of the Hall sensors. In this case, the measured Hall voltage U_{Hall}, mess is corrected by the offset voltage U_{Offset}. The offset voltages U_{Offset} of the Hall sensors are measured in a calibration process when the magnet is not in the vicinity of the Hall sensors. The measured Hall voltages U_{Hall,N} are corrected by the value of the offset voltage during evaluation. The Hall voltages U′_{Hall,N} specified above correspond to the corrected value of the Hall voltages. The situation depicted in FIG. 5 then arises again, namely that the Hall voltage is zero when the corresponding Hall sensor is situated exactly between the free ends of the permanent magnet.

It is possible and represents a preferred embodiment that such Hall sensors, which are momentarily not required for the determination of the position, are deactivated. In other words, the evaluation unit 34 stops measuring the Hall voltage until the measured value of the corresponding Hall sensor is needed again. To this end, it is possible for the Hall sensors to be divided into groups. If no Hall sensor of a corresponding group is used, the Hall sensors of the corresponding group are switched off.

FIG. 6 shows the damper 28. It should be noted that two permanent magnets 40.1, 40.2 are arranged on the piston 30. The Hall sensors 42.i are linearly arranged. Upon a movement of the piston 30 relative to the cylinder 32 and thus a movement of the cylinder 32 relative to the piston 30, the Hall sensors move along a trajectory T relative to the permanent magnets 40.1, 40.2. In the manner described above, the position of the piston 30 relative to the cylinder 32 can be adjusted with relatively high accuracy. The embodiment according to FIG. 1 enables an angular resolution of 0.01 degrees. The embodiment according to FIG. 6 enables an accuracy of approximately 0.1 mm.

Reference list

| | |
|---|---|
| 10 | aid |
| 12 | shaft |
| 14 | upper leg stump |
| 16 | lower leg |
| 20 | foot |
| 22 | cosmetic cover |
| 22 | swivel joint |
| 24 | movement element |
| 26 | reference element |
| 28 | damper |
| 30 | piston |
| 32 | cylinder |
| 34 | evaluation unit |
| 36 | actuator |
| 38 | position sensor |
| 40 | permanent magnet |
| 42 | Hall sensor |
| 44 | first magnet arm |
| 46 | second magnet arm |
| 48 | magnetic flux forming part |
| 50 | soft magnet element |
| 52 | non-ferromagnetic part |
| 54 | first partial permanent magnet |
| 56 | second partial permanent magnet |
| 58 | third partial permanent magnet |
| 60 | magnetic flux line curve |
| b | magnetic flux line |
| B_{N} | normal component |
| d | distance |
| E | sensor plane |
| E1 | first free end |
| E2 | second free end |
| G | straight line |
| H | height |
| i | running index |
| L | length |
| M | centre point |
| O₅₄ | first permanent magnet orientation |
| O₅₆ | second permanent magnet orientation |
| O₅₈ | third permanent magnet orientation |
| P | polarity |
| R | magnet arm direction |
| T | trajectory |
| U_{Hall} | Hall voltage |
| ΔU_{Hall} | Hall voltage difference |
| α | swivel angle |

1. An orthopaedic aid comprising:
a reference element;
a movement element, which is movably fixed to the reference element, and;
a position sensor for determining a position of the movement element relative to the reference element, the positive sensor comprising:
at least one permanent magnet and;
at least three Hall sensors;
wherein the Hall sensors are arranged on the reference element to move along a trajectory during a movement of the movement element relative to the reference element;
wherein the at least one permanent magnet is fixed to the movement element;
wherein the Hall sensors and the permanent magnet are arranged in such a way that a movement of the movement element relative to the reference element and a resulting movement of the Hall sensors along the trajectory effects a linear change in Hall voltage for at least one Hall sensor.

2. An orthopaedic aid comprising:
a reference element;
a movement element, which is movably fixed to the reference element;
a position sensor for determining a position of the movement element relative to the reference element, the position sensor comprising:
at least one permanent magnet;
at least three Hall sensors;
wherein the Hall sensors are arranged on the reference element and the movement element is arranged to move along a trajectory relative to the reference element;
wherein the at least one permanent magnet is fixed to the movement element;
wherein the at least one permanent magnet comprises:
a first magnet arm, which extends in a magnet arm direction;
a second magnet arm that is arranged at a distance from the first magnet arm and extends along the magnet arm direction;
the first magnet arm has a first free end with a first polarity;
the second magnet arm has a second free end with a second polarity opposite to the first polarity;
the free ends are arranged along the trajectory.

3. The orthopaedic aid according to claim 2, wherein the permanent magnet comprises:
a magnetic flux forming part;
that features a soft magnet element made of a magnetically soft material;
a first partial permanent magnet which:
forms the first magnet arm;
rests with its first contact end, which lies opposite the first free end, on the soft magnet element;
a second partial permanent magnet which:
forms the second magnet arm;
rests with its second contact end, which lies opposite the second free end, on the soft magnet element;
a third partial permanent magnet which:
is arranged between the first partial permanent magnet and the second partial permanent magnet;
extends transversely to the first magnet arm and the second magnet arm;
has a magnetic third permanent magnet orientation, which extends transversely to a first permanent magnet orientation of the first partial permanent magnet and extends transversely to a second permanent magnet orientation of the second partial permanent magnet.

4. The orthopaedic aid according to claim 1, wherein the magnetic flux forming part comprises a non-ferromagnetic part, which is arranged in at least one of the magnetic flux line curve between the first partial permanent magnet and the third partial permanent magnet, and in the magnetic flux line curve between the second partial permanent magnet and the third partial permanent magnet.

5. The orthopaedic aid according to claim 1, wherein the permanent magnet has a permanent magnet length a long the trajectory that is at least twice as great as a Hall sensor distance between two adjacent Hall sensors.

6. The orthopaedic aid according to claim 1, wherein a distance of the Hall sensors from the permanent magnet is at most half of the permanent magnet length.

7. The orthopaedic aid according to claim 1, further comprising an electric evaluation unit that is designed to automatically carry out a method featuring the steps:
detecting a first Hall voltage of a first Hall sensor and a second Hall voltage of a second Hall sensor that is adjacent to the first Hall sensor;
determining a position of the movement element relative to the reference element from a Hall voltage difference between the first Hall voltage and the second Hall voltage and from the second Hall voltage.

8. The orthopaedic aid according to claim 1, further comprising an electric evaluation unit that is designed to automatically carry out a method featuring the steps:
detecting the Hall voltages of at least three Hall sensors;
detecting the Hall sensors for which the Hall voltages assume the smallest values in terms of magnitude;
determining the position of the movement element relative to the reference element from the positions of these Hall sensors and a Hall voltage difference of these Hall voltages and the Hall voltage.

9. The orthopaedic aid according to claim 8, wherein the electric evaluation unit is designed to automatically execute a method containing the steps:
detecting the Hall sensor whose first Hall voltage indicates that the Hall sensor is situated in a homogeneous magnetic field;
determining a second Hall voltage of the Hall sensor that
is adjacent to this Hall sensor;
has a smaller Hall voltage in terms of magnitude than the other adjacent Hall sensor;
determining a position of the permanent magnet from a position of this Hall sensor and the Hall voltage difference between the first Hall voltage and the second Hall voltage and from the second Hall voltage.

10. The orthopaedic aid according to claim 1, wherein:
the reference element is a cylinder;
the movement element is a piston that is inside the cylinder;
the position sensor is a piston position sensor for measuring a position of the cylinder in the piston;
the permanent magnet is arranged on the piston;
the Hall sensors are arranged on the cylinder.

11. The orthopaedic aid according to claim 1, wherein:
the reference element is a first limb;
the movement element is a second limb;
the position sensor is a limb angle sensor for measuring an angular position of the first limb relative to the second limb.

12. The orthopaedic aid according to claim 1, wherein the evaluation unit is designed to switch off Hall sensors whose measurement results are not taken into account when calculating the position of the movement element.

13. A method for determining a position of an orthopaedic aid comprising:
a reference element;
a movement element;
a position sensor for determining a position of the movement element relative to the reference element, the position sensor comprising:
at least one permanent magnet;
at least three Hall sensors;
wherein the Hall sensors are arranged on the reference element and to move along a trajectory during a movement of the movement element relative to the reference element;
wherein the at least one permanent magnet is fixed to the movement element, with the steps:
detecting a first Hall voltage of a first Hall sensor and a second Hall voltage of a second Hall sensor that is adjacent to the first Hall sensor;
determining the position of the movement element relative to the reference element from a Hall voltage difference between the first Hall voltage and the second Hall voltage as well as from the second Hall voltage.

14. The method according to claim 13, further comprising the steps:
detecting the Hall voltages of at least three Hall sensors;
detecting the Hall sensors for which the Hall voltages assume the smallest values in terms of magnitude;
determining the position of the permanent magnet from the positions of these Hall sensors and the Hall voltage difference of these Hall voltages and the Hall voltage.

15. The method according to claim 13, further comprising the steps:
for each Hall sensor, detecting an offset voltage of the Hall voltage, which is caused by the existence of an angle between an actual position of the Hall sensor and a nominal position;
correcting the Hall voltage by the offset voltage.

16. The method according to claim 13, further comprising the steps:
for each Hall sensor, detecting a sensitivity that describes the dependency of the Hall voltage on the magnetic field;
correcting the position by the influence of the sensitivity.
